(19) United States
(12) Patent Application Publication
(10) Pub. No.: US 2009/0105524 A1
(43) Pub. Date: Apr. 23, 2009

(54) DEVICE FOR INDUCING BETTER SLEEP

(75) Inventors: CYNTHIA L. BRESSLER, Chappaqua, NY (US); Lisa Mercurio, New York, NY (US)

Correspondence Address: BLANK ROME LLP, WATERGATE, 600 NEW HAMPSHIRE AVENUE, N.W., WASHINGTON, DC, 20037, US

(73) Assignee: SMASHARTS LLC

(21) Appl. No.: 12/014,542

(22) Filed: Jan. 15, 2008

Related U.S. Application Data
(60) Provisional application No. 60/960,911, filed on Oct. 19, 2007.
(60) Provisional application No. 60/996,156, filed on Nov. 5, 2007.

(51) Int. Cl. A61M 21/02

(52) U.S. Cl. 600/27

(57) ABSTRACT

A device for inducing better sleep embedded with music having a tempo of about sixty to about eighty beats-per-minute. Listening to music with a tempo of approximately 60 to approximately 80 beats-per-minute results in significantly better sleep quality, better perceived sleep quality, longer sleep duration and greater sleep efficiency. Embodiments of the device can include, among other things, an alarm clock, a pillow, mobile, sleep mask, crib or a stuffed toy for use with children. Embodiments of the device including a travel clock, travel pillow, or mobile device allowing the device to be easily transported for purposes of inducing better sleep during travel are also contemplated.

The present application claims the benefit of U.S. Provisional Patent Application No. 60/960,911, filed Oct. 19, 2007; and U.S. Provisional Patent Application No. 60/996,156, filed Nov. 5, 2007, whose disclosures are hereby incorporated by reference in their entirety in the present application.

### BACKGROUND OF THE INVENTION

1. Field of Invention

The present invention relates generally to the field of electronic music devices. More particularly, the invention relates to a device for inducing better sleep.

2. Description of Related Art

Millions of people have difficulty falling asleep or staying asleep. Difficulty falling asleep may result stress, illness, or from disorders like insomnia. The effect of poor sleep can affect job performance and quality of life. The National Institutes of Health estimates that some 40 million people suffer from one or more chronic or long term sleep disorders in the United States alone, with another 20 million estimated to have frequent difficulty sleeping soundly at night and staying fully alert during the day.

Inducing sleep in children and infants is equally important and equally challenging. Similarly, elderly people, particularly those in the community living setting, often experience even greater difficulty sleeping than the general population. In addition, difficulty sleeping or falling asleep is often amplified when the sufferer is required to travel and/or attempt to sleep in an unfamiliar setting.

Several medicinal approaches have been developed for treating those who have difficulty sleeping and those who suffer from sleep disorders such as insomnia. Often times, however, doctors and sufferers of sleep difficulties are hesitant to pursue those approaches because of the addictive and residual effects that often accompany sleep medications. In addition, in the case of mild difficulty sleeping, prescription, and even non-prescription, steep medication often is not warranted.

A recent study on sleep quality was conducted by researchers at the Frances Payne Bolton School of Nursing of the Case Western Reserve University. The findings of that study provided evidence for the use of soothing music as an empirically-based intervention for sleep in older people. The study employed music having a tempo of 60 to 80 beats per minute.

While research has shown that soothing music can have a positive effect on sleep quality, there is a need for a mode of convenient and effective delivery of suitable music to a person suffering from difficulty sleeping for the purpose of inducing better sleep. The average person is not capable of identifying music having a tempo of only between about 60 and about 80 beats per minute. Further, a means for delivering music having only such a tempo is required. In addition, there is a need for a delivery mechanism for music having a tempo of only between 60 and 80 beats per minute that can be easily transported when a person is required to travel away from their usual place of sleep.

### SUMMARY AND OBJECTS OF THE INVENTION

Accordingly, there exists a need for a device for playing music having a tempo within a range of about sixty to about eighty beats-per-minute. There also exists a need for a device for playing such music that can be integrated into portable devices that can be easily transported during travel. Such devices should be of compact construction so as to be easily combined with common travel items.

Briefly described, those and other objects and features of the present invention are accomplished, as embodied and fully described herein, by a device for inducing sleep comprising a memory for storing digital music; a digital signal processor for processing the digital music; and a power source; wherein the digital music has a tempo within a range of about 60 to about 80 beats per minute.

The objects and features of the device also include a timer for playing the digital music for a predetermined length of time.

The objects and features of the device further include an actuator for activating the digital music.

The objects and features of the device also include at least one speaker for playing music.

The objects and features of the device also include at least one input for receiving digital music for storing to the memory.

With those and other objects, advantages and features of the invention that may become hereinafter apparent, the nature of the invention may be more clearly understood by reference to the following detailed description of the invention, the appended claims and to the several drawings attached herein.

### BRIEF DESCRIPTION OF THE DRAWINGS

FIG. 1 is a schematic drawing of the system architecture of the components of an embodiment of the present invention;

FIG. 2 is a drawing of an alarm clock embodiment of the present invention;

FIG. 3 is a drawing of a pillow embodiment of the present invention;

FIG. 4 is a drawing of a sleep mask embodiment of the present invention;

FIG. 5 is a drawing of a plush toy embodiment of the present invention;

FIG. 6 is a drawing of a mobile embodiment of the present invention;

FIG. 7 is a drawing of a crib embodiment of the present invention;

FIG. 8 is a drawing of an infant bouncy seat embodiment of the present invention;

FIG. 9 is a drawing of a baby monitor embodiment of the present invention;

FIG. 10 is a process flow diagram depicting an embodiment of the present invention.

### DETAILED DESCRIPTION OF THE PREFERRED EMBODIMENT

Several preferred embodiments of the invention are described for illustrative purposes, it being understood that the invention may be embodied in other forms not specifically shown in the drawings.

While the digitized music as described in the preferred embodiments is referred to as “Bedtime Beats” music, it is understood that the invention contemplates any music having a tempo of between about 60 and about 80 beats per minute.

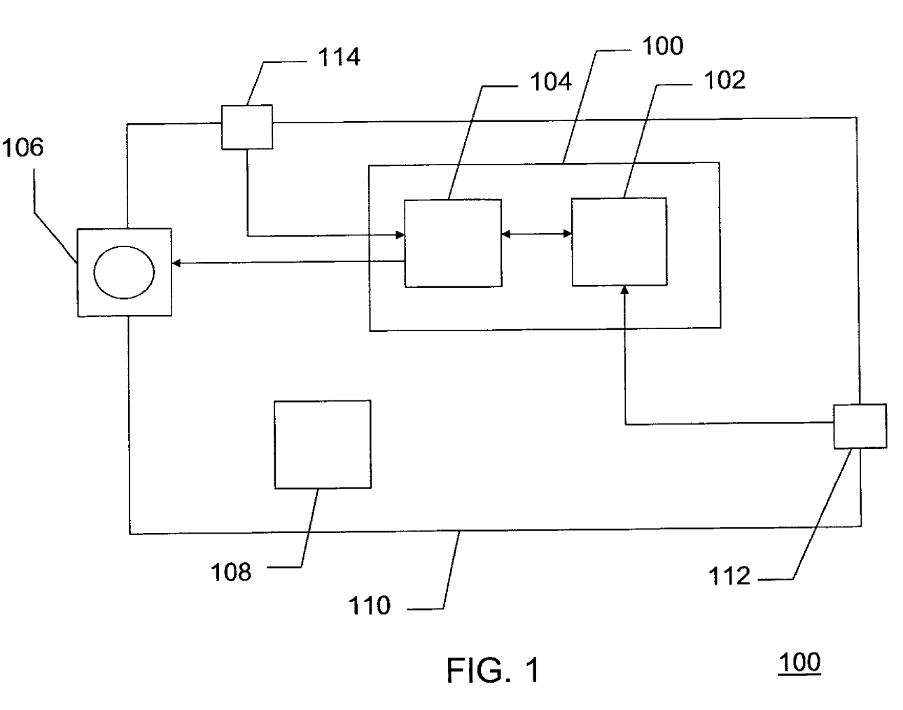

Turning to FIG. 1, the embodiments of the present invention comprise a chip 100 having a memory 102 for storing digitized music, a digital audio signal processor 104 for processing the digitized music, an output component such as a speaker 106 for outputting the digitized music and a power source 108 for supplying power to the system. Embodiments further comprise a housing 110 which can take on varying forms as would occur to a person of ordinary skill in the art, select examples of which are described in more detail below. The housing 110 may include an input 112 for receiving digital music to be stored to the memory 102 and an actuator 114 for beginning playback of the digital music.

Bedtime Beats Home Clock:

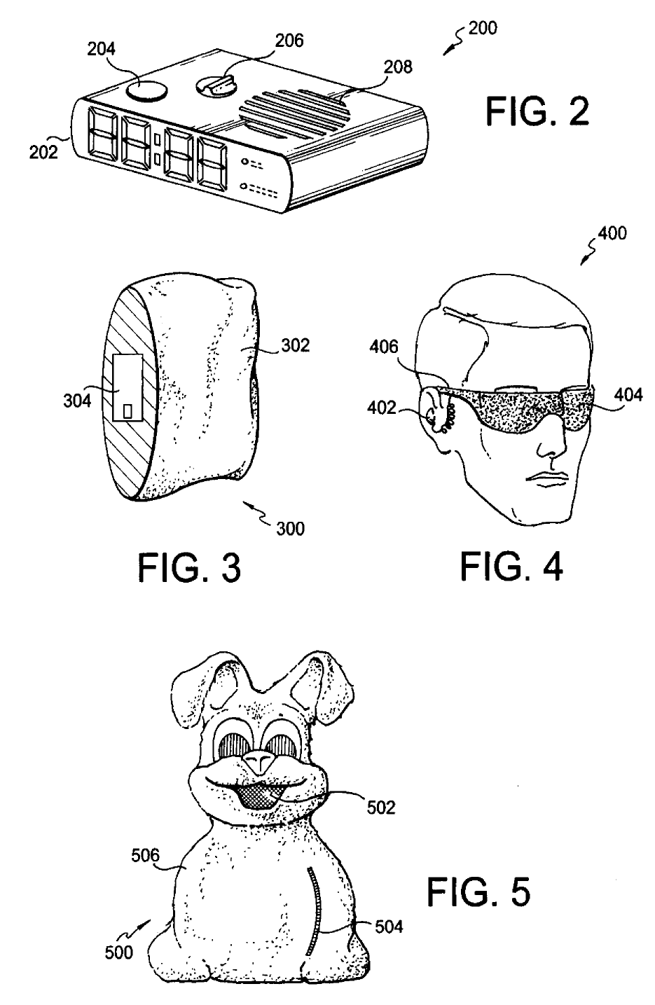

In one embodiment of the present invention as shown in FIG. 2, a clock 200 is embedded with 120 minutes of Bedtime Beats music, or other predeterminable length of time as desired. The clock housing 202 may feature a “Secret to Sleep” button or other music actuator feature 204 that the user may use to start playback of the Bedtime Beats music. Upon activation, the Bedtime Beats music will play for the predetermined length of time. The clock may also feature an adjustable timer 206 and the user may be able to select the length of the music's playing time (for example, 120, 60, 30, 15 minutes, or other length of time as desired). The chip containing the Bedtime Beats music is housed inside the clock 200. The clock has a speaker 208 for playing the music for the sleeper. In varying embodiments, the clock housing may contain the features of an alarm clock, a “fall asleep” clock, a personal music device docking station, or any combination of these features, or other features of a clock that would occur to a person of ordinary skill in the art.

This technology may be featured in similar types of device housings such as clock radios, portable audio devices, home audio devices, and docking devices for portable audio and/or video devices (e.g., iHome docking device for Apple iPod), as will be apparent to those of ordinary skill in the art.

Bedtime Beats Travel Clock:

In another embodiment of the present invention, the clock as previously described may be portable and may contain the same features as the Home Clock and may include 60 minutes of music, or other length of time as desired.

Bedtime Beats Standard Size Pillow:

In another embodiment of the present invention, a pillow 300 is embedded with 60 minutes of Bedtime Beats music, or other length of time as desired. The pillow housing 302 may be made of therapeutic foam or other soft material and may contain a protective compartment 304 to hold the chip, speaker and power source. The pillow 300 may contain an actuator for beginning playback of the music such as by squeezing the pillow 300.

Bedtime Beats Travel Pillow:

In another embodiment of the present invention, a travel pillow may be similar to the standard size pillow 300 previously described but having a smaller size to facilitate easy transport during travel. The exterior may be covered in soft fleece or other soft material.

Bedtime Beats Sleep Mask:

Turning to FIG. 4, shown therein is a sleep mask embodiment 400 with ear bud speakers 402 and an embedded chip containing 30 minutes of Bedtime Beats music, or other length of time as desired. The sleep mask 400 may have a piece of fabric, padding, or other material 404 for covering the eyes and an elastic band 406 or other mechanism for securing the mask over the eyes of the wearer. The mask may have ear bud speakers 402 attached to the fabric material 404 or, alternatively, to the elastic band 406 which allow the wearer to listen to the Bedtime Beats music while wearing the sleep mask 400.

Bedtime Beats Toy:

In another embodiment of the present invention as shown in FIG. 5, a very soft, washable plush toy (“Sleepie”) 500 is embedded with a speaker 502 and the chip containing 30 minutes of Bedtime Beats music, or other length of time as desired. The speaker 502 and chip may be placed in a protective compartment 504 and may be removable so that the Sleepie 500 can be washed. The Sleepie 500 may include an actuator 506 for beginning playback of the Bedtime Beats music by some action by the user such as squeezing the Sleepie 500. The toy may be in the form of a stuffed animal, plush ball, a soft/fabric book, or other soft toy for infants. The toy may also be in the form of a stationary infant toy such as a song box or other toy that may be placed on a child's dresser or other location near the child's place of sleep.

Bedtime Beats Mobile:

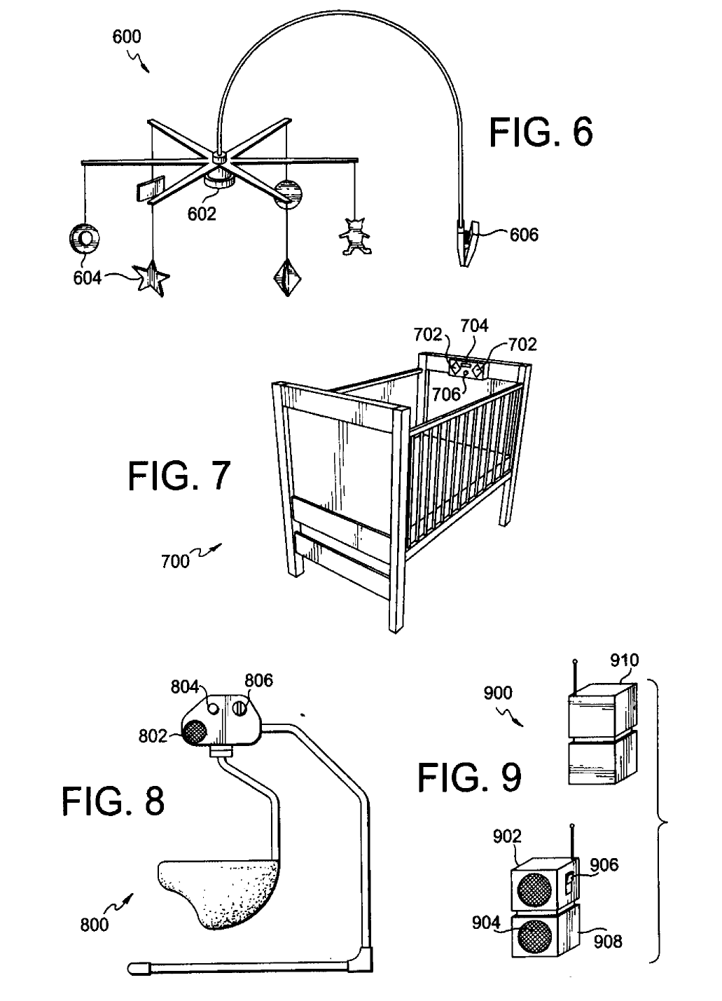

Turning to FIG. 6, in another embodiment of the present invention, a musical mobile 600 is embedded with a speaker 602 and a chip containing 30 minutes of Bedtime Beats music, or other length of time as desired. The mobile 600 may contain 6 small, rotating toys 604, or such other number as desired. The toys 604 may be constructed of a plush material or other material and designed to take the form of animals, shapes, vehicles, or other forms. The mobile 600 may have a clip 606 for attaching it to a crib. In another embodiment, the mobile may include a stand so that it may rest on a dresser, desk, or other flat surface.

Bedtime Beats Crib:

In another embodiment of the present invention as shown in FIG. 7, a crib 700 is embedded with speakers 702 and a chip containing 30 minutes of Bedtime Beats music, or other length of time as desired. The crib 700 may feature a “Secret to Sleep” button 704 or other music activation feature for starting playback of the Bedtime Beats music. The crib 700 may also feature an adjustable timer 706 and a user may be able to select the length of the music's playing time (for example, 120, 60, 30, 15 minutes, or other length of time as desired).

Bedtime Beats Baby Swing Bouncy Seat:

In another embodiment of the present invention as shown in FIG. 8, shown therein is an infant bouncy seat or swing 800 inside of which a child rests. Typically positioned on the floor, the seat or swing is moveable. The baby swing 800 generally swings back and forth creating a rocking motion and can be battery operated or plugged in to a wall outlet. The swing 800 is embedded with speakers 802 and a chip containing 30 minutes of Bedtime Beats music, or other length of time as desired. The baby swing 800 may feature a “Secret to Sleep” button 804 or other music activation feature for starting playback of the Bedtime Beats music. The baby swing 800 may also feature an adjustable timer 806 and a user may be able to select the length of the music's playing time (for example, 120, 60, 30, 15 minutes, or other length of time as desired).

Similar to the baby swing 800, the infant bouncy seat is a stand alone infant seat which contains a seat for the infant to sit inside of. The infant's legs are free to touch and push off the floor to create a bouncing motion. Bedtime Beats music is embedded in this device as in the infant bouncy seat 800.

Bedtime Beats Baby Monitor:

Another embodiment of the present inventions is shown in FIG. 9, wherein a baby monitor 900 is embedded with a chip containing 30 minutes of Bedtime Beats music, or other length of time as desired. Typically, a baby monitor consists of a pair of speaker/microphone combination devices, one in the parental bedroom and one in the child's bedroom that allows a parent to hear, speak to, and monitor the child remotely. The Bedtime Beats baby monitor housing 902 contains a speaker 904 which acts as both a speaker for the parents to monitor and hear the child and for administering of Bedtime Beats music which can play from the parental unit 908 on the child's speaker 904. The child's unit 910 will also house the Bedtime Beats chip and can play music when an actuator 906 is switched over to “play” function. The units may be battery powered or plugged in to a standard wall outlet. The parental unit 908 and the child unit 910 may be of identical design to allow for interchangeability.

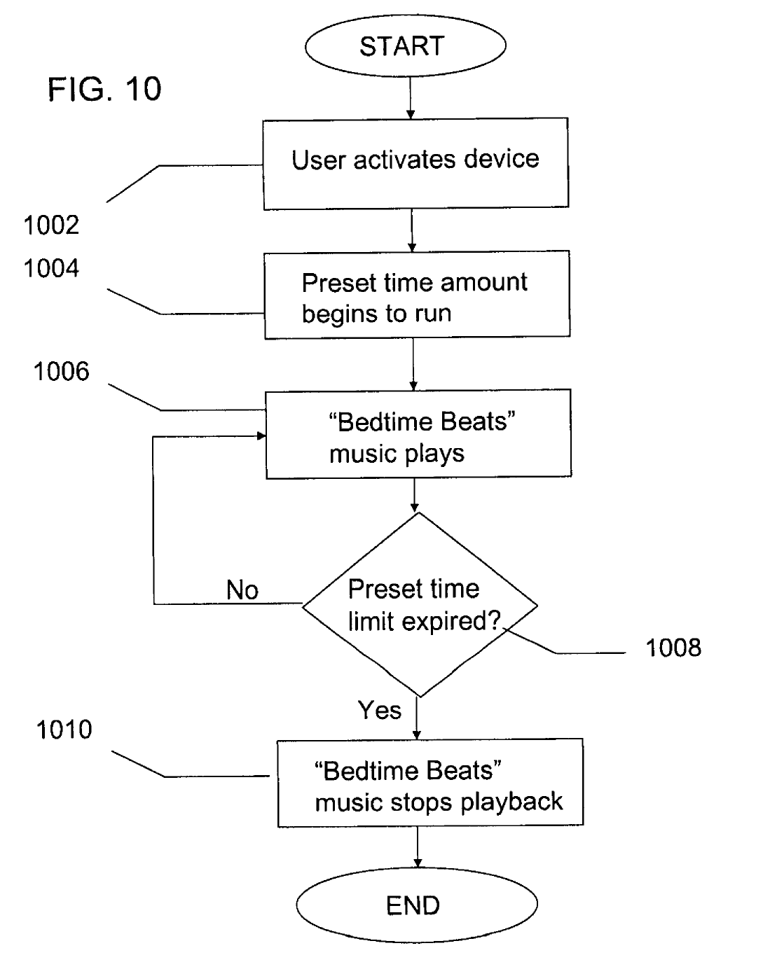

Turning to FIG. 10, described therein is the process flow 1000 of the Bedtime Beats chip. In process step 1002, a user activates the Bedtime Beats device. In process step 1004, a preset timer begins to run. In process step 1006, the Bedtime Beats music begins to play. In process step 1008, if time remains on the timer, the music continues to play. If time has expired, the Bedtime Beats stops playing as shown in process step 1010.

Although certain presently preferred embodiments of the disclosed invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

We claim:

1. A device for inducing sleep comprising:
a housing a memory for storing digital music;
a digital signal processor for processing the digital music; and
a power source;
wherein the digital music has a tempo within a range of about 60 to about 80 beats per minute.

2. The device for inducing sleep according to claim 1, further comprising a timer for playing the digital music for a predetermined length of time.

3. The device for inducing sleep according to claim 1, further comprising an actuator for activating the digital music.

4. The device for inducing sleep according to claim 3, wherein the actuator is a button located on an outer surface of the housing.

5. The device for inducing sleep according to claim 3, wherein the actuator is a sensor responsive to pressure located in the interior of the housing that a user may activate by squeezing the device.

6. The device for inducing sleep according to claim 1, wherein the housing is a clock.

7. The device for inducing sleep according to claim 1, wherein the housing is a pillow.

8. The device for inducing sleep according to claim 1, wherein the housing is a toy, the toy comprising:
a layer of soft padding;
a soft exterior layer covering the layer of soft padding; and
an interior cavity for containing the device.

9. The device for inducing sleep according to claim 8, wherein the toy is washable.

10. The device for inducing sleep according to claim 1, wherein the housing is a mobile, which further comprises a plurality of rotating items;

11. The device for inducing sleep according to claim 1, further comprising at least one speaker for playing music.

12. The device for inducing sleep according to claim 1, further comprising a button for starting playback of the digital music.

13. The device for inducing sleep according to claim 1, wherein the housing is a sleep mask.

14. The device for inducing sleep according to claim 1, wherein the housing is a crib.

15. The device for inducing sleep according to claim 1, wherein the housing is an infant bouncy seat.

16. The device for inducing sleep according to claim 1, wherein the housing is a baby monitor.

17. The device for inducing sleep according to claim 2, wherein the timer is adjustable for adjusting the predetermined length of time.

18. The device for inducing sleep according to claim 1, further comprising an input for receiving digital music for storing in the memory.

19. The device for inducing sleep according to claim 1, wherein the device is portable.
